(19) United States
(12) Patent Application Publication
(10) Pub. No.: US 2013/0060346 A1
(43) Pub. Date: Mar. 7, 2013

(54) Hip Prosthesis System

(71) Applicant: Simon Collins, Tetbury (GB)

(72) Inventor: Simon Collins, Tetbury (GB)

(73) Assignee: CORIN LIMITED, GLOUCESTERSHIRE (GB)

(21) Appl. No.: 13/698,661

(22) PCT Filed: Jan. 19, 2011
(86) PCT No.: PCT/GB11/50085

§ 371 Date: Nov. 18, 2012

(30) Foreign Application Priority Data
May 18, 2010 (GB) 1008233.7

(51) Int. Cl. A61F 2/36

(52) U.S. Cl. 623/23.12

(57) ABSTRACT

A hip prosthesis system (10) comprises a hip-resurfacing femoral component (12) for use with a prepared natural femoral neck, a total-hip-replacement femoral component (14) for insertion into the femur following removal of the natural head and at least in part neck and which is different to the hip-resurfacing femoral component (12), and a common femoral cap (16) which defines a part-spherical articulating surface (82) for engagement with an acetabular cup and which is selectively engagably mountable on the hip-resurfacing femoral component (12) and the total-hip-replacement femoral component (14). A method of intra-operably selecting between a hip resurfacing procedure and a total hip replacement procedure using such a system (10) is also provided.

The present invention relates to a hip prosthesis system and method particularly for use with a hip-resurfacing procedure and a total-hip replacement procedure.

A type of hip joint replacement is determined prior to surgery, and this may typically be either total hip arthroplasty or hip-resurfacing arthroplasty. Surgery is then prepared based on this decision, and the necessary prosthesis is organised.

However, once surgery begins, and particularly during trialling, it can become clear to the surgeon that the bone quality at the patient's hip may have deteriorated far more than anticipated during pre-surgery diagnosis, or vice versa that the bone quality is actually far better than initially thought. This then causes difficulties for the surgeon since different prosthetic component parts, and even prosthetic parts for a different type of arthroplasty are not readily available.

Furthermore, with the present state of the art, should it be decided intra-operatively that a different kind of hip arthroplasty is required, then the current procedure must be terminated and a full set of different hip prosthetic components for the different kind of arthroplasty must be obtained and the trialling and fitment procedures restarted, leading to a significant wastage of time.

Additionally, ordering and stocking complete sets of prosthetic components for two completely different kinds of hip arthroplasty is costly in terms of materials and space.

The present invention seeks to provide a solution to these problems.

According to a first aspect of the present invention, there is provided a hip prosthesis system comprising a hip-resurfacing femoral component for use with a prepared natural femoral neck, a total-hip-replacement femoral component for insertion into the femur following removal of the natural head and at least in part neck and which is different to the hip-resurfacing femoral component, and a common femoral cap which defines a part spherical articulating surface for engagement with an acetabular cup and which is selectively engagably mountable on the hip-resurfacing femoral component and the total-hip-replacement femoral component.

Preferable and/or optional features of the first aspect of the invention are set forth in claims 2 to 21, inclusive.

According to a second aspect of the present invention, there is provided a method of intra-operably selecting between a hip resurfacing procedure and a total hip replacement procedure, the method comprising the step of providing a hip-resurfacing femoral component, a total-hip-replacement femoral component, and a common femoral cap; trialling the hip-resurfacing femoral component and common femoral cap with a patient; and intra-operatively substituting the hip-resurfacing femoral component for the total-hip-replacement femoral component whilst retaining the common femoral cap and dependent on a discovered bone-quality of the patient.

Preferably, a plurality of differently sized common femoral caps, a plurality of differently sized hip-resurfacing components and/or a plurality of differently sized total-hip-replacement femoral components are provided for intra-operative selection.

The invention will now be more particularly described, by way of example only, with reference to the accompanying drawing, in which:

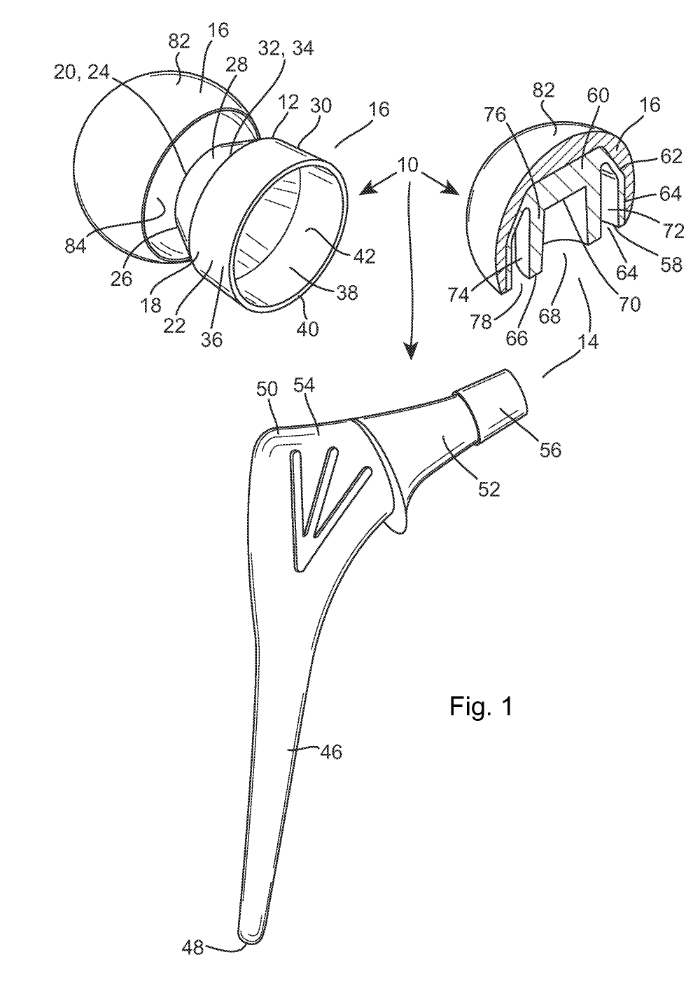

FIG. 1 shows component parts of one embodiment of a hip prosthesis system, in accordance with the first aspect of the invention, a first adaptor of a hip-resurfacing femoral component being shown in spaced relationship with a common femoral cap, and a second adaptor of a total-hip-replacement femoral component being shown in cross-section for clarity and mated with the common femoral cap which is also shown in cross-section.

Referring to the sole FIG. 1, a hip prosthesis system 10 is shown which comprises a first hip-resurfacing femoral component 12, a second total-hip-replacement femoral component 14, and a common femoral cap 16. The hip-resurfacing femoral component 12 includes a first adaptor 18 which comprises a head portion 20 and a contiguous wall 22 which extends from the head portion 20. In this embodiment, the head portion 20 is a uniform planar or substantially planar circular or substantially circular element 24 having a preferably chamfered perimeter edge 26. The wall 22 extends uniformly and continuously from the chamfered perimeter edge 26.

The wall 22 includes a frusto-conical or substantially frusto-conical proximal wall portion 28 which is contiguous with the head portion 20 and which extends at a first angle, typically being between 60 degrees and 85 degrees, to the plane of the head portion 20.

A frusto-conical or substantially frusto-conical distal wall portion 30 which is contiguously formed at a distal edge 32 of the proximal wall portion 28, extends at a second angle which is different to the first angle. In this case, the second angle is typically between 75 and 90 degrees to the plane of the head portion 20. The mating edge 34 between the proximal and distal wall portions 28, 30 is again preferably smoothly chamfered to reduce any sharpness. The distal wall portion 30 may have an axial length which thus provides a depending skirt 36 when in use and suitable for accommodating leg length deficiency. In this case, the thickness of the head portion 20 may be altered to provide for the depending skirt. This will be reference again further hereinbelow.

The head portion 20 and wall 22 define a femoral-neck cavity 38 for receiving a resected and prepared natural femoral neck of a femur. Although the exterior surface of the wall 22 is adapted to preferably provide two different tapers in an axial direction from the free tail edge 40 to the head portion 20, the interior surface 42 defining the femoral-neck cavity 38 may have two different tapers from the free tail edge 40 to the inner surface of the head portion 20, a single taper, or no taper. In this latter case, the interior surface 42 may be cylindrical or substantially cylindrical, or may be multi-faceted.

The interior surface 42 of the femoral-neck cavity 38 may also include one or more axial and/or lateral adhesive channels, which may be rectilinear and/or arcuate, for receiving a bone bonding agent. Additionally or alternatively, the interior surface 42 may include one or more inwardly projecting splines for bone engagement with a femoral neck. Generally axially facing pins may protrude from the inner surface of the head portion.

As a further option, in addition or as an alternative to the options above, the femoral-neck cavity 38 may include one or more apertures through the wall 22 and/or head portion 20 for receiving bone screws or other suitable fasteners to more reliably engage the first adaptor 18 with the prepared natural femoral neck. The fastener may be formed of resorbable material to allow the first adaptor 18 to be more readily assimilated with the original bone and to improve bone density in the region of the hip-resurfacing femoral component 12 over time. Each aperture may include an outer recessed portion for receiving a head of the fastener in a, preferably flush, recessed fashion. A, preferably integral, head-retaining element may preferably be included in the recess, such as a radially inwardly projecting lip, to prevent or limit the fastener from backing out once the head is received therebelow.

In a modification of the first adaptor 18 of the hip-replacement femoral component, the head portion 20 may be part-spherical. To this end, the contiguous wall may only include the proximal wall portion which may also project to provide a skirt, as discussed above. The proximal wall portion may be convergingly tapering in the direction towards the head portion 20 from the plane of its free tail edge. Alternatively, the proximal wall portion may be cylindrical or substantially cylindrical.

In this modification, the femoral-neck cavity 38 may extend into or be at least in part defined by the head portion 20, and thus itself may be part-spherical at least at and adjacent to the head portion 20.

In all other respects, this modified first adaptor of the hip-resurfacing femoral component can be or include the features as described above.

The first adaptor may or may not include a projecting shaft which extends, typically centrally, from the femoral-neck cavity. The shaft may be removable or formed as one-piece with the first adaptor, and is typically rectilinear but tapering.

Beneficially, a plurality of first hip-resurfacing femoral components 12 is provided, having different dimensions and sizes, to enable greater intra-operative selection. However, the exterior surface of the first adaptor 18 is common to enable connection with the common femoral cap 16.

The total-hip-replacement femoral component 14 preferably comprises a prosthetic tapering elongate femoral stem 46 defining a distal pointed or rounded tail end 48 and a proximal shoulder end 50, a prosthetic neck 52 extends from the shoulder 54 of the femoral stem 46, and a trunnion 56 is formed at the end of the neck 52 opposite the shoulder end 50. The femoral stem 46, neck 52 and trunnion 56 can be formed as one-piece. However, the neck 52 and trunnion 56 may be one-piece which is separable of the femoral stem 46, thereby enabling interchangability between femoral stems and necks with trunnions. Furthermore, the necks 52 and trunnions 56 may be separate parts, again enabling greater interchangeability to match specific patient requirements.

Depending on the kind of total-hip-replacement femoral component 14, the shoulder 54 may be pronounced relative to the rest of the stem 46, as shown in FIG. 1, or may be smoothly integrated so as to be less pronounced as the stem transitions to the neck. The shoulder may also simply be formed as a flat or plateau laterally to the longitudinal axis of the stem. The femoral component may include a traditional or larger style of stem, and/or may include a commonly known ‘mini-stem’.

The femoral stem 46 itself may have a significant longitudinal extent so that it extends into the femoral bone, past the intertrochanteric line and well along the shaft of the femur, or it may be a so-called ‘mini-stem’ which only extends a short distance in the femoral bone beyond the intertrochanteric line.

The total-hip-replacement femoral component 14 also includes a second adaptor 58 for interfacing between the trunnion 56 and the common femoral cap 16. The second adaptor 58 has a head portion 60 and a contiguous wall 62 which extends from the head portion 60, similarly to the first adaptor 18. The exterior 64 of the second adaptor 58 thus matches that of the first adaptor 18 as described above, inclusive of the suggested possible optional features, and therefore further detailed description is omitted.

The head portion 60 and the wall 62 define a trunnion cavity 64 for receiving the trunnion 56 extending from the neck 52 provided on the femoral stem 46. The trunnion cavity 64, in this case, includes a boss 66 having a bore 68. The boss 66 is formed as one-piece with the head portion 60 on its interior surface 70. As such, an interior surface 72 of the wall 62 is spaced from an exterior surface 74 of a wall 76 of the boss 66. This is beneficial in reducing material usage and improving lightness of the component.

The bore 68 of the boss 66 is preferably tapered to complimentarily match the trunnion 56 as a sliding fit. As such, a Morse taper fit can be provided between the trunnion 56 and the second adaptor 58. However, other means of engagement can be considered. For example, the boss bore 68 may be cylindrical or substantially cylindrical, and a fastening element, such as a surgical screw or grub-screw can be utilised to hold the trunnion 56 and second adaptor 58 together. Another example would be to utilise mating screw-threads on the trunnion 56 and the boss bore 68, or interference fit axial splines and spline channels.

The gap 78 between the exterior surface 74 of the boss 66 and the interior surface 72 of the trunnion cavity 64 can be dispensed with so as to provide a solid head portion 60, whereby the trunnion cavity 64 only comprises the bore 68 for receiving the trunnion 56. To this end, suitable fasteners as described with regard to the first adaptor 18 above can optionally be utilised to secure the second adaptor 58 to the trunnion 56.

As will be appreciated, it is preferable that a plurality of different total-hip-replacement femoral components 14 of different sizes and kinds are provided for greater intra-operable selection by a surgeon.

The common femoral cap 16 in selective combination with the first adaptor 18 and the second adaptor 58 forms the prosthetic femoral head. The femoral cap 16 defines a part-spherical articulating surface 82 for articulating engagement with a part-spherical articulating surface of an acetabular cup, typically also being a prosthetic device.

The femoral cap 16 includes an adaptor cavity 84 which typically extends along the polar axis of the part-spherical outer surface 82. The adaptor cavity 84 is shaped to be a tolerance complementary fit with the head portions 20, 60 and at least part of the walls 22, 62 of the first and second adaptors 18, 58. Generally, at least one of the tapering proximal or distal wall portions 28, 30 of the first and second adaptors 18, 58 will provide a taper engagement with the adaptor cavity 84 of the femoral cap 16. As such, the exterior surfaces of the head portions 20, 60 of the first and second adaptors 18, 58 may not engage, contact or abut the interior surface of the adaptor cavity 84 of the femoral cap 16.

Preferably, a plurality of femoral caps 16 having the common adaptor cavity 84 is provided so that outer part-spherical articulating surfaces 82 of different dimensions and sizes can be utilised with both the hip-resurfacing femoral component 12 and the total-hip-replacement femoral component 14.

In use, surgery is prepared with one or more differently sized common femoral caps 16 having the uniformly sized adaptor cavity 84, one or more differently sized hip-resurfacing femoral components 12, and one or more differently sized total-hip-replacement femoral components 14. Once the operation begins, the surgeon can better judge which of the hip-resurfacing procedure and the total-hip-replacement procedure will better suit the condition of the patient's revealed femur and acetabulum. The surgeon may trial the hip-resurfacing femoral component 12 and a suitable common femoral cap 16. However, the surgeon may then decide, intra-operatively, that the bone structure is not suitable for the hip-resurfacing procedure, for example, due to bone degeneration in the femoral head and neck. Whilst retaining the selected common femoral cap 16 due to its suitability with the acetabular component and the patient's acetabular structure, the surgeon can immediately switch to the total-hip-replacement procedure. As such, the total-hip-replacement femoral component 14 can be trialled and implanted whilst retaining the already trialled and selected common femoral cap 16. Due to the common adaptor cavity 84 of the femoral cap 16, connection to the total-hip-replacement femoral component 14 via the second adaptor 58 instead of utilising the hip-resurfacing femoral component 12 and the first adaptor 18 is simple and straightforward.

Preferably, the common femoral cap 16 has a modulus of elasticity which is greater than that of the hip-resurfacing femoral component 12 and the total-hip-replacement femoral component 14. The common femoral cap 16 is preferably ceramic, or includes an outer surface layer which is ceramic mounted on a metal or plastics supporting layer. Other hard bearing biocompatible materials can be considered and used, but as of the priority date of the invention, ceramic is preferred.

The hip-resurfacing femoral component 12 and the total-hip-replacement femoral component 14 are preferably formed from metal, such as titanium alloy or cobalt chromium alloy. However, if the femoral components, and in particular the total-hip-replacement femoral component 14, are more modular in nature, then different materials can be considered to improve workability, cost-savings, implantation, and ultimately use by the patient once implanted.

Each hip-resurfacing femoral component and/or total-hip-replacement femoral component can include a common femoral cap pre-attached thereto pre-surgery.

It is feasible that the first adaptor 18 can be formed of a resorbable material. The resorbable material, by way of example, can be or include polyglycolic acid, polylactic resorbable polymers, co-polymers of these or other known bio resorbable materials including metals such as magnesium alloys.

All or part of a surface which contacts original bone of a femur may be provided with an osteo-conductive coating, such as hydroxyapetite, Titanium Plasma coating or metallic beaded type surface for use in a cementless implant procedure. As mentioned above, the hip-resurfacing femoral component, total-hip-replacement femoral component or parts thereof may be attached using a suitable bone cement.

By providing the first and second adaptors with common head portion and wall exterior surfaces, a single adaptor cavity enables a common femoral cap to be utilised with two different kinds of femoral component which are associated with two completely different kinds of hip arthroplasty procedure. As such, the present invention facilitates quick and simple intra-operative cross-procedure interchangability of prosthetic components.

The hip prosthesis system of the present invention allows the surgeon many benefits. Firstly, the system offers the surgeon the intra-operative choice of being able to decide that if the bone quality of the patient at the time of surgery is insufficient, he can then choose to revert to a total hip arthroplasty utilising the second adaptor with the common femoral cap. Further, dependent on the amount of bone available in the acetabular socket, the surgeon may choose to opt intra-operatively for a total hip arthroplasty to be able to then make the acetabular socket smaller and preserve bone. Additionally, the hip-resurfacing femoral component allows the surgeon to better visualise the acetabular socket during preparation, subsequently fitting the common femoral cap to the selected adaptor. Additional benefits of the system of the present invention are that the surgeon may intra-operatively decide that, after making bone cuts, the leg length may be reduced by proceeding with the resurfacing option. If it is discovered during the surgical procedure that a patient has an, or an excessive, leg length deficiency, since it is known that leg length deficiency is a risk factor for hip resurfacing and as such may create an imbalance leading to premature failure of the prosthesis, the surgeon can opt to revert to total hip arthroplasty utilising the common femoral cap. The hip prosthesis system of the present invention also has advantages in that it can allow a hospital to carry less stock and parts, and in light of private hospitals and National Health Service trusts aiming to provide more services with increasingly restricted budgets, allows hospital storage space and costs to be reduced. Furthermore, materials and manufacturing costs are reduced.

The embodiments described above are provided by way of examples only, and various other modifications will be apparent to persons skilled in the art without departing from the scope of the invention as defined by the appended claims.

1. A hip prosthesis system comprising a hip-resurfacing femoral component for use with a prepared natural femoral neck, a total-hip-replacement femoral component which is insertable into a femur following removal of the natural head and at least in part neck and which is different to the hip-resurfacing femoral component, and a common femoral cap which defines a part spherical articulating surface which is engagable with an acetabular cup and which is selectively engagably mountable on the hip-resurfacing femoral component and the total-hip-replacement femoral component.

2. A hip prosthesis system as claimed in claim 1, wherein a plurality of differently sized common femoral caps is provided.

3. A hip prosthesis system as claimed in claim 1, wherein a plurality of differently sized hip-resurfacing femoral components and/or differently sized total-hip-replacement femoral components are provided.

4. A hip prosthesis system as claimed in claim 1, wherein the hip-resurfacing femoral component includes a first adaptor on which the common femoral cap is mountable.

5. A hip prosthesis system as claimed in claim 4, wherein the first adaptor of the hip-resurfacing femoral component is adapted to receive a prepared natural femoral neck of a femur.

6. A hip prosthesis system as claimed in claim 5, wherein the first adaptor includes a head portion and a wall which extends from the head portion, the wall being complementarily shaped to engage with an interior surface of the common femoral cap.

7. A hip prosthesis system as claimed in claim 6, wherein the wall includes a proximal portion at or adjacent to the head portion at a first angle relative to the head portion, and a distal portion which is spaced from the head portion so that the proximal portion is interposed therebetween and which is at a different second angle relative to the head portion.

8. (canceled)

9. (canceled)

10. A hip prosthesis system as claimed in claim 7, wherein the distal portion of the wall is adapted, upon engagement, to protrude from the common femoral cap to adjust leg deficiency.

11. (canceled)

12. A hip prosthesis system as claimed in claim 5, wherein the first adaptor includes a part-spherical head portion and a wall portion which extends from the head portion, the head portion being complementarily shaped to engage with an interior surface of the common femoral cap.

13. (canceled)

14. A hip prosthesis system as claimed in claim 5, wherein a second adaptor of the total-hip-replacement femoral component is adapted to receive a prosthetic modular hip-stem trunnion.

15. A hip prosthesis system as claimed in claim 1, wherein the total-hip-replacement femoral component comprises an elongate femoral stem which is embeddable in the femur after removal of at least the femoral head, and a trunnion which extends at an angle to a longitudinal extent of the elongate femoral stem, the trunnion being adapted to support the common femoral cap.

16. A hip prosthesis system as claimed in claim 15, wherein the total-hip-replacement femoral component further comprises a second adaptor on which the common femoral cap is mountable so as to be indirectly supportable by the trunnion.

17. A hip prosthesis system as claimed in claim 16, wherein the second adaptor is slidably engagable with the trunnion.

18. A hip prosthesis system as claimed in one of claim 1, wherein the common femoral cap is attachable pre-surgery to the hip-resurfacing femoral component or the total-hip-replacement femoral component.

19. A hip prosthesis system as claimed in claim 1, wherein the common femoral cap has a modulus of elasticity which is greater than that of the hip-resurfacing femoral component and the total-hip-replacement femoral component.

20. A hip prosthesis system as claimed in the claim 1, wherein the common femoral cap is or includes ceramic.

21. A hip prosthesis system as claimed in claim 1, wherein the hip-resurfacing femoral component and total-hip-replacement femoral component are formed of a first material, and the common femoral cap is formed of a second material which is different to that of the first material.

22. (canceled)

23. A method of intra-operably selecting between a hip resurfacing procedure and a total hip replacement procedure, the method comprising the step of providing a hip-resurfacing femoral component, a total-hip-replacement femoral component, and a common femoral cap; trialling the hip-resurfacing femoral component and common femoral cap with a patient; and intra-operatively substituting the hip-resurfacing femoral component for the total-hip-replacement femoral component or vice versa whilst retaining the common femoral cap and dependent on a discovered bone-quality of the patient.

24. A method as claimed in claim 23, wherein a plurality of differently sized common femoral caps, a plurality of differently sized hip-resurfacing components and/or a plurality of differently sized total-hip-replacement femoral components are provided for intra-operative selection.

25. A method as claimed in claim 24, utilising a hip prosthesis system comprising a hip-resurfacing femoral component for use with a prepared natural femoral neck, a total-hip-replacement femoral component which is insertable into the femur following removal of the natural head and at least in part neck and which is different to the hip-resurfacing femoral component, and a common femoral cap which defines a part spherical articulating surface which is engagable with an acetabular cup and which is selectively engagably mountable on the hip-resurfacing femoral component and the total-hip-replacement femoral component.
